(19) United States
(12) Patent Application Publication
(10) Pub. No.: US 2024/0398991 A1
(43) Pub. Date: Dec. 5, 2024

(54) AAVRH74 PARTICLES FOR GENE THERAPY OF MUSCLE DISEASE

(71) Applicant: University of Florida Research Foundation, Incorporated, Gainesville, FL (US)

(72) Inventors: Arun Srivastava, Gainesville, FL (US); Keyun Qing, Gainesville, FL (US); Jakob Shoti, Gainesville, FL (US)

(73) Assignee: University of Florida Research Foundation, Incorporated, Gainesville, FL (US)

(21) Appl. No.: 18/692,438

(22) PCT Filed: Sep. 16, 2022
(86) PCT No.: PCT/US2022/076588

§ 371 Date: Mar. 15, 2024

Related U.S. Application Data
(60) Provisional application No. 63/244,757, filed on Sep. 16, 2021.

(51) Int. Cl. A61K 48/00; C07K 14/005; C12N 15/86

(52) U.S. Cl. CPC A61K 48/0058; C07K 14/005; C12N 15/86; C12N 2750/14122; C12N 2750/14143

(57) ABSTRACT

Provided herein are AAVrh74 capsid proteins comprising one or more amino acid substitutions or deletions that confer a liver-detargeting property to AAV particles comprising them. Provided herein are AAVrh74 capsid proteins comprising one or more amino acid substitutions that confer an improved transduction efficiency (e.g., in muscle cells). Also provided herein are methods of using AAVrh74 capsid proteins comprising one or more amino acid substitutions or deletions.

### RELATED APPLICATIONS

This application claims priority under 35 U.S.C. § 119 (e) to U.S. Provisional Application Ser. No. 63/244,757, entitled “AAVRH74 PARTICLES FOR GENE THERAPY OF MUSCLE DISEASE”, filed on Sep. 16, 2021, the contents of which are incorporated herein by reference in their entirety.

### REFERENCE TO AN ELECTRONIC SEQUENCE LISTING

The contents of the electronic sequence listing (U120270080WO00-SEQ-COB.xml; Size: 31,501 bytes; and Date of Creation: Sep. 12, 2022) are herein incorporated by reference in their entirety.

### BACKGROUND

Gene therapy has the potential to treat subject suffering from or are at risk of suffering from genetic disease. Improved AAV vectors for carrying genetic payload would be beneficial to the development of gene therapies, e.g., for certain diseases that affect muscle tissue and/or function. Muscle diseases, such as muscular dystrophies, can result from numerous conditions including, for example, congenital or acquired somatic mutations, injury, and exposure to hazardous compounds. In some cases, muscle diseases result in life-threatening complications or lead to serious symptoms and/or death. Although numerous factors have been implicated in regulating muscle diseases, including muscular dystrophies, effective treatments remain limited.

### SUMMARY

The present disclosure is based at least in part on the realization that delivery of gene/s (e.g., a therapeutic gene) to many tissues and/or organs in the body (e.g., muscle tissue) using adeno-associated virus (AAV) particles is difficult because a substantial portion of AAV particles administered to a subject get sequestered by the subject's liver, effectively reducing the AAV particles available to deliver gene to target tissues and/or organs. As a strategy to overcome this difficulty, the present disclosure provides AAV capsid proteins comprising one or more amino acid substitutions (in some embodiments, including deletions) and particles comprising them that are liver de-targeted, i.e. sequestered by a subject liver to a lesser degree relative to AAV particles not comprising the one or more amino acid substitutions.

Herein, this concept is further explained in the context of AAV particles of serotype rh74 for targeting muscle tissue. However, it should be understood that compositions and methods provided herein as a means of liver-detargeting of an AAV particle, can be applied to an AAV or any serotype and to target any tissue. In the context of targeting muscle tissue for gene delivery, provided herein is an AAV capsid protein comprising one or more amino acid substitutions. In some embodiments, provided herein is an AAVrh74 capsid protein comprising an amino acid deletion or substitution at T265.

In some aspects, provided herein is an AAV capsid protein (e.g., an AAVrh74 capsid protein) comprising an amino acid substitution or deletion at a position corresponding to T265 of wild-type AAVrh74 capsid protein, e.g., of SEQ ID NO: 1. In some embodiments, an AAV capsid protein (e.g., an AAVrh74 capsid protein) further comprises an amino acid substitution at a position corresponding to Y447, T494, K547, N665, and/or Y733 of wild-type AAVrh74 capsid protein, e.g., of SEQ ID NO: 1. In some embodiments, one or more amino acid deletions and/or substitutions in the capsid protein of an AAVrh74 capsid protein confers to the particle comprising it a greater transduction efficiency with respect to a particular type of cells (e.g., muscle cells).

In some embodiments, an amino acid corresponding to T265 of wild-type AAVrh74 capsid protein of SEQ ID NO: 1 is deleted. In some embodiments, an amino acid corresponding to T265 of wild-type AAVrh74 capsid protein of SEQ ID NO: 1 is substituted. In some embodiments, a substitution at the amino acid position corresponding to T265 of wild-type AAVrh74 capsid protein of SEQ ID NO: 1 is T265D, T265F, or T265G.

In some embodiments, an AAV capsid protein (e.g., AAVrh74 capsid protein) comprises an amino acid substitution or deletion at a position corresponding to T265 and amino acid substitutions at positions corresponding to: (a) Y447 and Y733; or (b) Y447, Y733, and T494 of wild-type AAVrh74 capsid protein of SEQ ID NO: 1. In some embodiments, a capsid protein comprises a deletion or one of the following substitutions at a position corresponding to T265: T265D. T265F, and T265G, and substitutions corresponding to: (a) Y447F and Y733F; or (b) Y447F. Y733F, and T494T of wild-type AAVrh74 capsid protein of SEQ ID NO: 1.

In some aspects, the present disclosure provides a nucleic acid encoding any one of the AAV capsid proteins (e.g., AAV1, AAV2, AAV3, AAV4, AAV5, AAV6, AAV7, AAV8, AAV9, AAV10, AAVrh10 AAV11, AAV12, AAV13, or AAVrh74 capsid proteins) having one or more deletions or amino acid substitutions as provided herein.

In some aspects, provided herein is an AAV particle comprising any one of the AAV capsid proteins (e.g., AAV1, AAV2, AAV3, AAV4, AAV5, AAV6, AAV7, AAV8, AAV9, AAV10, AAVrh10 AAV11, AAV12, AAV13, or AAVrh74 capsid proteins) having one or more deletions or amino acid substitutions as provided herein. In some embodiments, an AAV particle as provided herein comprises a nucleic acid that is encapsidated by any one of the AAV capsid proteins (e.g., AAV1, AAV2, AAV3, AAV4, AAV5, AAV6, AAV7, AAV8, AAV9, AAV10, AAVrh10 AAV11, AAV12, AAV13, or AAVrh74 capsid proteins) having one or more deletions or amino acid substitutions as provided herein. In some embodiments, a nucleic acid encapsidated by any one of the capsid proteins as provided herein comprises a gene of interest. In some embodiments, a nucleic acid encapsidated by any one of the capsid proteins as provided herein comprises a promoter, such as a muscle-specific promoter. In some embodiments, a gene of interest encodes a therapeutic protein. In some embodiments, a therapeutic protein is dystrophin, myotilin, lamin, caveolin, caplain-3, dysferlin, a sarcoglycan, TCAP, TRIM32, FKRP, titin, acetylflucosamine epimerase, Desmin, LARGE, fukutin, an integrin, salenoprotein, a collagen, plectin, or a functional fragment thereof.

In some aspects, provided herein is a composition comprising any one of the AAV particles as provided herein. In some embodiments, a composition of AAV particles comprises a pharmaceutically acceptable carrier.

In some aspects, provided herein is a method comprising administering to a subject any one of the compositions comprising AAV particles as described herein. In some embodiments, a subject is human. In some embodiments, a subject suffers from or is at risk of suffering from a muscular dystrophy.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein. It is to be understood that the data illustrated in the drawings in no way limit the scope of the disclosure.

FIG. 1A shows sequence alignment of capsid proteins of AAV2, AAV8 and AAVrh74. FIG. 1A shows that Y447, Y733, and T494 are conserved in AAVrh74. FIG. 1B shows that T265 is conserved in AAVrh74 capsid protein.

FIG. 2 exemplifies a strategy of deleting or substituting T265 in AAVrh74.

FIG. 3 provides a schematic showing a strategy of deleting or substituting T265 in AAVrh74 either alone (left panel) or in combination with a double-mutant (DM, middle panel) or triple-mutant (TM, right panel). The DM constitutes Y447+733F. The TM constitutes Y447+733F+T494V.

FIGS. 4A-4C show quantifications of bioluminescence imaging studies. FIG. 4A shows bioluminescence signal quantified from whole body bioluminescence imaging of mice administered wild-type (WT) ssAAVrh74-Fluc particles. FIG. 4B shows bioluminescence signal quantified in the liver from whole body bioluminescence imaging of mice administered WT ssAA Vrh74-Fluc particles or T265del-ssAA Vrh74-Fluc particles. FIG. 4C shows bioluminescence signal quantified in the muscle from whole body bioluminescence imaging of mice administered WT ssAAVrh74-Fluc particles or T265del-ssAAVrh74-Fluc particles.

### DETAILED DESCRIPTION

Provided herein are compositions and methods useful for detargeting AAV particles from the liver, i.e. reducing their tropism for the liver, or reducing the portion of a composition of AAV particles administered to a subject from being sequestered by the subject's liver. In some embodiments of the strategy provided here, one or more amino acids in a capsid protein (e.g., VP1, VP2 and/or VP3) of a capsid protein are substituted or deleted. Deletions and/or substitutions of amino acids in a capsid protein may lead also to improved transduction efficiency in certain cells/tissues (e.g., muscle cells/tissue). The AAV capsid proteins, particles comprising them, compositions comprising the particles can be used in a variety of applications including but not limited to methods of treating a subject suffering from or at risk of suffering from a disease or disorder (e.g., a muscular dystrophy) by delivering one or more genes of interest to a particular tissue or organ.

### Capsid Proteins

Provided herein are AAV capsid proteins having one or more amino acid deletions or substitutions. In some embodiments, an AAV capsid protein is of serotype 1, 2, 3, 4, 5, 6, 7, 8, 9, 10, rh10, 11, 12, 13, or rh74. Example amino acid sequences of these AAVs are provided in SEQ ID NOs: 1-14. SEQ ID NO: 15 provides an example of a nucleic acid sequence encoding an AAVrh74 capsid protein. In some embodiments, an AAV capsid protein as provided herein is an AAVrh74 capsid protein. An AAV capsid protein may be a VP1, VP2, or VP3 capsid protein.

In some embodiments, the one or more amino acid deletions or substitutions as provided herein confer/s to the AAV particle that comprises the capsid protein an improved liver detargeting ability, improved tropism for a particular tissue type (e.g., muscle tissue), and/or improved transduction efficiency in particular cells or tissue type (e.g., muscle cells or tissue).

In some embodiments, an AAV (e.g., AAVrh74) capsid protein comprises an amino acid substitution or deletion at a position that corresponds to T265 of wild-type AAVrh74. SEQ ID NO: 1 provides an example of an amino acid sequence of AAVrh74 capsid protein. The different capsid proteins VP1, VP2, and VP3 are defined according to numbering of the full-length VP1 protein. In some embodiments, for AAVrh74 capsid proteins, a VP1 capsid protein is defined by amino acids 1-738 of SEQ ID NO: 1; a VP2 capsid protein is defined by amino acids 138-738 of SEQ ID NO: 1; and a VP3 capsid protein is defined by amino acids 204-738 of SEQ ID NO: 1. Numbering of AAV capsid proteins is provided according to the VP1 sequence. For example, Y447 refers to the tyrosine at position 447 of SEQ ID NO: 1 in a VP1 protein or the corresponding tyrosine in a VP2 or VP3 protein. Similarly, T494, K547, N665, and Y733 refer to the threonine at position 494, lysine at position 547, asparagine at position 665, and tyrosine at position 733 of SEQ ID NO: 1, respectively, in a VP1 protein, or the corresponding amino acids in a VP2 or VP3 protein.

### Example of an Amino Acid Sequence of AAVrh74 Capsid Protein:

```
(SEQ ID NO: 1)
    1 MAADGYLPDW LEDNLSEGIR EWWDLKPGAP KPKANQQKQD NGRGLVLPGY
  51 KYLGPFNGLD KGEPVNAADA AALEHDKAYD QQLQAGDNPY LRYNHADAEF
101 QERLQEDTSF GGNLGRAVFQ AKKRVLEPLG LVESPVKTAP GKKRPVEPSP
151 QRSPDSSTGI GKKGQQPAKK RLNFGQTGDS ESVPDPQPIG EPPAGPSGLG
201 SGTMAAGGGA PMADNNEGAD GVGSSSGNWH CDSTWLGDRV ITTSTRTWAL
251 PTYNNHLYKQ ISNGISGGST NDNTYFGYST PWGYFDENRF HCHFSPRDWQ
301 RLINNNWGFR PKRLNFKLEN IQVKEVTQNE GTKTIANNLT STIQVFTDSE
351 YQLPYVLGSA HQGCLPPFPA DVEMIPQYGY LTLNNGSQAV GRSSFYCLEY
401 FPSQMLRIGN NFEFSYNFED VPFHSSYAHS QSLDRLMNPL IDQYLYYLSR
451 TQSTGGTAGT QQLLFSQAGP NNMSAQAKNW LPGPCYRQQR VSTTLSQNNN
501 SNFAWTGATK YHLNGRDSLV NPGVAMATHK DDEERFFPSS GVLMFGKQGA
551 GKDNVDYSSV MLTSEEEIKT TNPVATEQYG VVADNLQQQN AAPIVGAVNS
601 QGALPGMVWQ NRDVYLQGPI WAKIPHTDGN FHPSPLMGGF GLKHPPPQIL
651 IKNTPVPADP PTTFNQAKLA SFITQYSTGQ VSVEIEWELQ KENSKRWNPE
701 IQYTSNYYKS TNVDFAVNTE GTYSEPRPIG TRYLTRNL
```

### Example of an Amino Acid Sequence of Wild-Type AAV1 Capsid Protein

```
(SEQ ID NO: 2)
    1 MAADGYLPDW LEDNLSEGIR EWWDLKPGAP KPKANQQKQD DGRGLVLPGY
  51 KYLGPFNGLD KGEPVNAADA AALEHDKAYD QQLKAGDNPY LRYNHADAEF
101 QERLQEDTSF GGNLGRAVFQ AKKRVLEPLG LVEEGAKTAP GKKRPVEQSP
151 QEPDSSSGIG KTGQQPAKKR LNFGQTGDSE SVPDPQPLGE PPATPAAVGP
201 TTMASGGGAP MADNNEGADG VGNASGNWHC DSTWLGDRVI TTSTRTWALP
251 TYNNHLYKQI SSASTGASND NHYFGYSTPW GYFDFNRFHC HFSPRDWQRL
301 INNNWGFRPK RLNFKLENIQ VKEVTINDGV TTIANNLIST VQVFSDSEYQ
351 LPYVLGSAHQ GCLPPFPADV FMIPQYGYLT LNNGSQAVGR SSFYCLEYEP
401 SQMLRTGNNF TFSYTFEEVP FHSSYAHSQS LDRLMNPLID QYLYYLNRTQ
451 NQSGSAQNKD LLFSRGSPAG MSVQPKNWLP GPCYRQQRVS KTKTDNNNSN
501 FTWTGASKYN LNGRESIINP GTAMASHKDD EDKFFPMSGV MIFGKESAGA
551 SNTALDNVMI TDEEEIKATN PVATERFGTV AVNFQSSSTD PATGDVHAMG
601 ALPGMVWQDR DVYLQGPIWA KIPHTDGHFH PSPLMGGFGL KNPPPQILIK
651 NTPVPANPPA EFSATKFASF ITQYSTGQVS VEIEWELQKE NSKRWNPEVQ
701 YTSNYAKSAN VDFTVDNNGL YTEPRPIGTR YLTRPL
```

### Example of an Amino Acid Sequence of Wild-Type AAV2 Capsid Protein

```
(SEQ ID NO: 3)
    1 MAADGYLPDW LEDTLSEGIR QWWKLKPGPP PPKPAERHKD DSRGLVLPGY
  51 KYLGPFNGLD KGEPVNEADA AALEHDKAYD RQLDSGDNPY LKYNHADAEF
101 QERLKEDTSF GGNLGRAVFQ AKKRVLEPLG LVEEPVKTAP GKKRPVEHSP
151 VEPDSSSGTG KAGQQPARKR LNFGQTGDAD SVPDPQPLGQ PPAAPSGLGT
201 NTMATGSGAP MADNNEGADG VGNSSGNWHC DSTWMGDRVI TTSTRTWALP
251 TYNNHLYKQI SSQSGASNDN HYFGYSTPWG YFDENRFHCH FSPRDWQRLI
301 NNNWGFRPKR LNFKLFNIQV KEVTQNDGTT TIANNLTSTV QVFTDSEYQL
351 PYVLGSAHQG CLPPFPADVF MVPQYGYLTL NNGSQAVGRS SFYCLEYFPS
401 QMLRIGNNFT FSYTFEDVPF HSSYAHSQSL DRLMNPLIDQ YLYYLSRINT
451 PSGTTTQSRL QFSQAGASDI RDQSRNWLPG PCYRQQRVSK TSADNNNSEY
501 SWTGATKYHL NGRDSLVNPG PAMASHKDDE EKFFPQSGVL IFGKQGSEKT
551 NVDIEKVMIT DEEEIRTTNP VATEQYGSVS TNLQRGNRQA ATADVNTQGV
601 LPGMVWQDRD VYLQGPIWAK IPHTDGHFHP SPLMGGFGLK HPPPQILIKN
651 TPVPANPSTT FSAAKFASFI TQYSTGQVSV EIEWELQKEN SKRWNPEIQY
701 TSNYNKSVNV DFTVDINGVY SEPRPIGTRY LTRNL
```

### Example of an Amino Acid Sequence of Wild-Type AAV3 Capsid Protein

```
(SEQ ID NO: 4)
    1 MAADGYLPDW LEDNLSEGIR EWWALKPGVP QPKANQQHQD NRRGLVLPGY
  51 KYLGPGNGLD KGEPVNEADA AALEHDKAYD QQLKAGDNPY LKYNHADAEF
101 QERLQEDTSF GGNLGRAVFQ AKKRILEPLG LVEEAAKTAP GKKGAVDQSP
151 QEPDSSSGVG KSGKQPARKR LNFGQTGDSE SVPDPQPLGE PPAAPTSLGS
201 NTMASGGGAP MADNNEGADG VGNSSGNWHC DSQWLGDRVI TISTRTWALP
251 TYNNHLYKQI SSQSGASNDN HYFGYSTPWG YFDENRFHCH FSPRDWQRLI
301 NNNWGFRPKK LSFKLFNIQV RGVTQNDGTT TIANNLISTV QVFTDSEYQL
351 PYVLGSAHQG CLPPFPADVF MVPQYGYLTL NNGSQAVGRS SFYCLEYFPS
401 QMLRIGNNFQ FSYTFEDVPF HSSYAHSQSL DRLMNPLIDQ YLYYLNRTQG
451 TTSGTTNQSR LLFSQAGPQS MSLQARNWLP GPCYRQQRLS KTANDNNNSN
501 FPWTAASKYH LNGRDSLVNP GPAMASHKDD EEKFFPMHGN LIFGKEGTTA
551 SNAELDNVMI TDEEEIRTIN PVATEQYGTV ANNLQSSNTA PTTGTVNHQG
601 ALPGMVWQDR DVYLQGPIWA KIPHTDGHFH PSPLMGGFGL KHPPPQIMIK
651 NTPVPANPPT TFSPAKFASF ITQYSTGQVS VEIEWELQKE NSKRWNPEIQ
701 YTSNYNKSVN VDFTVDINGV YSEPRPIGTR YLTRNL
```

### Example of an Amino Acid Sequence of Wild-Type AAV4 Capsid Protein

```
(SEQ ID NO: 5)
    1 MTDGYLPDWL EDNLSEGVRE WWALQPGAPK PKANQQHQDN ARGLVLPGYK
  51 YLGPGNGLDK GEPVNAADAA ALEHDKAYDQ QLKAGDNPYL KYNHADAEFQ
101 QRLQGDTSFG GNLGRAVFQA KKRVLEPLGL VEQAGETAPG KKRPLIESPQ
151 QPDSSTGIGK KGKQPAKKKL VFEDETGAGD GPPEGSTSGA MSDDSEMRAA
201 AGGAAVEGGQ GADGVGNASG DWHCDSTWSE GHVTTTSTRT WVLPTYNNHL
251 YKRLGESLQS NTYNGFSTPW GYFDFNRFHC HFSPRDWQRL INNNWGMRPK
301 AMRVKIFNIQ VKEVTTSNGE TTVANNLIST VQIFADSSYE LPYVMDAGQE
351 GSLPPFPNDV FMVPQYGYCG LVTGNTSQQQ TDRNAFYCLE YFPSQMLRTG
401 NNFEITYSFE KVPFHSMYAH SQSLDRLMNP LIDQYLWGLQ STTTGTTLNA
451 GTATTNFTKL RPTNFSNFKK NWLPGPSIKQ QGFSKTANQN YKIPATGSDS
501 LIKYETHSTL DGRWSALTPG PPMATAGPAD SKFSNSQLIF AGPKQNGNTA
551 TVPGTLIFTS EEELAATNAT DTDMWGNLPG GDQSNSNLPT VDRLTALGAV
601 PGMVWQNRDI YYQGPIWAKI PHTDGHFHPS PLIGGFGLKH PPPQIFIKNT
651 PVPANPATTF SSTPVNSFIT QYSTGQVSVQ IDWEIQKERS KRWNPEVQFT
701 SNYGQQNSLL WAPDAAGKYT EPRAIGTRYL THHL
```

### Example of an Amino Acid Sequence of Wild-Type AAV5 Capsid Protein

```
(SEQ ID NO: 6)
    1 MSFVDHPPDW LEEVGEGLRE FLGLEAGPPK PKPNQQHQDQ ARGLVLPGYN
  51 YLGPGNGLDR GEPVNRADEV AREHDISYNE QLEAGDNPYL KYNHADAEFQ
101 EKLADDTSFG GNLGKAVFQA KKRVLEPFGL VEEGAKTAPT GKRIDDHFPK
151 RKKARTEEDS KPSTSSDAEA GPSGSQQLQI PAQPASSLGA DTMSAGGGGP
201 LGDNNQGADG VGNASGDWHC DSTWMGDRVV TKSTRTWVLP SYNNHQYREI
251 KSGSVDGSNA NAYFGYSTPW GYFDFNRFHS HWSPRDWQRL INNYWGFRPR
301 SLRVKIFNIQ VKEVTVQDST TTIANNLIST VQVFTDDDYQ LPYVVGNGTE
351 GCLPAFPPQV FTLPQYGYAT LNRDNTENPT ERSSFFCLEY FPSKMLRIGN
401 NFEFTYNFEE VPFHSSFAPS QNLFKLANPL VDQYLYRFVS TNNTGGVQFN
451 KNLAGRYANT YKNWFPGPMG RTQGWNLGSG VNRASVSAFA TTNRMELEGA
501 SYQVPPQPNG MINNLQGSNT YALENTMIEN TYLEGNMLIT TYLEGNMLIT
551 SESETQPVNR VAYNVGGQMA TNNQSSTTAP ATGTYNLQEI VPGSVWMERD
601 VYLQGPIWAK IPETGAHFHP SPAMGGFGLK HPPPMMLIKN TPVPGNITSF
651 SDVPVSSFIT QYSTGQVTVE MEWELKKENS KRWNPEIQYT NNYNDPQFVD
701 FAPDSTGEYR TTRPIGTRYL TRPL
```

### Example of an Amino Acid Sequence of Wild-Type AAV6 Capsid Protein

```
(SEQ ID NO: 7)
    1 MAADGYLPDW LEDNLSEGIR EWWDLKPGAP KPKANQQKQD DGRGLVLPGY
  51 KYLGPENGLD KGEPVNAADA AALEHDKAYD QQLKAGDNPY LRYNHADAEF
101 QERLQEDTSF GGNLGRAVFQ AKKRVLEPFG LVEEGAKTAP GKKRPVEQSP
151 QEPDSSSGIG KTGQQPAKKR LNFGQTGDSE SVPDPQPLGE PPATPAAVGP
201 TTMASGGGAP MADNNEGADG VGNASGNWHC DSTWLGDRVI TTSTRTWALP
251 TYNNHLYKQI SSASTGASND NHYFGYSTPW GYFDFNRFHC HFSPRDWQRL
301 INNNWGFRPK RLNFKLFNIQ VKEVTINDGV TTIANNLIST VQVFSDSEYQ
351 LPYVLGSAHQ GCLPPFPADV FMIPQYGYLT LNNGSQAVGR SSFYCLEYFP
401 SQMLRTGNNF TFSYTFEDVP FHSSYAHSQS LDRLMNPLID QYLYYLNRTQ
451 NQSGSAQNKD LLFSRGSPAG MSVQPKNWLP GPCYRQQRVS KTKTDNNNSN
501 FTWTGASKYN LNGRESIINP GTAMASHKDD KDKFFPMSGV MIFGKESAGA
551 SNTALDNVMI TDEEEIKATN PVATERFGTV AVNLQSSSTD PATGDVHVMG
601 ALPGMVWQDR DVYLQGPIWA KIPHTDGHFH PSPLMGGFGL KHPPPQILIK
651 NTPVPANPPA EFSATKFASF ITQYSTGQVS VEIEWELQKE NSKRWNPEVQ
701 YTSNYAKSAN VDFTVDNNGL YTEPRPIGTR YLTRPL
```

### Example of an Amino Acid Sequence of Wild-Type AAV7 Capsid Protein

```
(SEQ ID NO: 8)
    1 MAADGYLPDW LEDNLSEGIR EWWDLKPGAP KPKANQQKQD NGRGLVLPGY
  51 KYLGPENGLD KGEPVNAADA AALEHDKAYD QQLKAGDNPY LRYNHADAEF
101 QERLQEDTSF GGNLGRAVFQ AKKRVLEPLG LVEEGAKTAP AKKRPVEPSP
151 QRSPDSSTGI GKKGQQPARK RLNFGQTGDS ESVPDPQPLG EPPAAPSSVG
201 SGTVAAGGGA PMADNNEGAD GVGNASGNWH CDSTWLGDRV ITTSTRTWAL
251 PTYNNHLYKQ ISSETAGSIN DNTYFGYSTP WGYFDFNRFH CHFSPRDWQR
301 LINNNWGFRP KKLRFKLFNI QVKEVTINDG VITIANNLIS TIQUESDSEY
351 QLPYVLGSAH QGCLPPFPAD VFMIPQYGYL TLNNGSQSVG RSSFYCLEYF
401 PSQMLRTGNN FEFSYSFEDV PFHSSYAHSQ SLDRLMNPLI DQYLYYLART
451 QSNPGGTAGN RELQFYQGGP STMAEQAKNW LPGPCFRQQR VSKILDQNNN
501 SNFAWTGATK YHLNGRNSLV NPGVAMATHK DDEDRFFPSS GVLIFGKTGA
551 TNKTTLENVL MTNEEEIRPT NPVATEEYGI VSSNLQAANT AAQTQVVNNQ
601 GALPGMVWQN RDVYLQGPIW AKIPHTDGNF HPSPLMGGFG LKHPPPQILI
651 KNTPVPANPP EVFTPAKFAS FITQYSTGQV SVEIEWELQK ENSKRWNPEI
701 QYTSNFEKQT GVDFAVDSQG VYSEPRPIGT RYLTRNL
```

### Example of an Amino Acid Sequence of Wild-Type AAV8 Capsid Protein

```
(SEQ ID NO: 9)
    1 MAADGYLPDW LEDNLSEGIR EWWALKPGAP KPKANQQKQD DGRGLVLPGY
  51 KYLGPFNGLD KGEPVNAADA AALEHDKAYD QQLQAGDNPY LRYNHADAEF
101 QERLQEDTSF GGNLGRAVFQ AKKRVLEPLG LVEEGAKTAP GKKRPVEPSP
151 QRSPDSSTGI GKKGQQPARK RLNFGQTGDS ESVPDPQPLG EPPAAPSGVG
201 PNTMAAGGGA PMADNNEGAD GVGSSSGNWH CDSTWLGDRV ITTSTRTWAL
251 PTYNNHLYKQ ISNGTSGGAT NDNTYFGYST PWGYFDENRF HCHFSPRDWQ
301 RLINNNWGFR PKRLSFKLEN IQVKEVTQNE GTKTIANNLT STIQVFTDSE
351 YQLPYVLGSA HQGCLPPFPA DVFMIPQYGY LTLNNGSQAV GRSSFYCLEY
401 FPSQMLRTGN NFQFTYTFED VPFHSSYAHS QSLDRLMNPL IDQYLYYLSR
451 TQTTGGTANT QTLGFSQGGP NTMANQAKNW LPGPCYRQQR VSTTTGQNNN
501 SNFAWTAGTK YHLNGRNSLA NPGIAMATHK DDEERFFPSN GILIFGKQNA
551 ARDNADYSDV MLTSEEEIKT TNPVATEEYG IVADNLQQQN TAPQIGTVNS
601 QGALPGMVWQ NRDVYLQGPI WAKIPHTDGN FHPSPLMGGF GLKHPPPQIL
651 IKNTPVPADP PTTFNQSKLN SFITQYSTGQ VSVEIEWELQ KENSKRWNPE
701 IQYTSNYYKS TSVDFAVNTE GVYSEPRPIG TRYLTRNL
```

### Example of an Amino Acid Sequence of Wild-Type AAV9 Capsid Protein

```
(SEQ ID NO: 10)
    1 MAADGYLPDW LEDNLSEGIR EWWALKPGAP QPKANQQHQD NARGLVLPGY
  51 KYLGPGNGLD KGEPVNAADA AALEHDKAYD QQLKAGDNPY LKYNHADAEF
101 QERLKEDTSF GGNLGRAVFQ AKKRLLEPLG LVEEAAKTAP GKKRPVEQSP
151 QEPDSSAGIG KSGAQPAKKR LNFGQTGDTE SVPDPQPIGE PPAAPSGVGS
201 LTMASGGGAP VADNNEGADG VGSSSGNWHC DSQWLGDRVI TTSTRTWALP
251 TYNNHLYKQI SNSTSGGSSN DNAYFGYSTP WGYFDENRFH CHFSPRDWQR
301 LINNNWGFRP KRLNFKLFNI QVKEVTDNNG VKTIANNLTS TVQVFTDSDY
351 QLPYVLGSAH EGCLPPFPAD VEMIPQYGYL TLNDGSQAVG RSSFYCLEYE
401 PSQMLRIGNN FQFSYEFENV PFHSSYAHSQ SLDRLMNPLI DQYLYYLSKT
451 INGSGQNQQT LKFSVAGPSN MAVQGRNYIP GPSYRQQRVS TTVTQNNNSE
501 FAWPGASSWA LNGRNSLMNP GPAMASHKEG EDRFFPLSGS LIFGKQGTGR
551 DNVDADKVMI TNEEEIKTTN PVATESYGQV ATNHQSAQAQ AQTGWVQNQG
601 ILPGMVWQDR DVYLQGPIWA KIPHTDGNFH PSPLMGGFGM KHPPPQILIK
651 NTPVPADPPT AFNKDKLNSF ITQYSTGQVS VEIEWELQKE NSKRWNPEIQ
701 YTSNYYKSNN VEFAVNTEGV YSEPRPIGTR YLTRNL
```

### Example of an Amino Acid Sequence of Wild-Type AAV10 Capsid Protein

```
(SEQ ID NO: 11)
    1 MAADGYLPDW LEDNLSEGIR EWWDLKPGAP KPKANQQKQD DGRGLVLPGY
  51 KYLGPENGLD KGEPVNAADA AALEHDKAYD QQLKAGDNPY LRYNHADAEF
101 QERLQEDTSF GGNLGRAVFQ AKKRVLEPLG LVEEGAKTAP GKKRPVEPSP
151 QRSPDSSTGI GKKGQQPAKK RLNFGQTGDS ESVPDPQPIG EPPAGPSGLG
201 SGTMAAGGGA PMADNNEGAD GVGSSSGNWH CDSTWLGDRV ITTSTRTWAL
251 PTYNNHLYKQ ISNGISGGST NDNTYFGYST PWGYFDENRF HCHFSPRDWQ
301 RLINNNWGFR PKRLNFKLEN IQVKEVTQNE GTKTIANNLT STIQVFTDSE
351 YQLPYVLGSA HQGCLPPFPA DVFMIPQYGY LTLNNGSQAV GRSSFYCLEY
401 FPSQMLRTGN NFEFSYQFED VPFHSSYAHS QSLDRLMNPL IDQYLYYLSR
451 TQSTGGTAGT QQLLFSQAGP NNMSAQAKNW LPGPCYRQQR VSTTLSQNNN
501 SNFAWTGATK YHLNGRDSLV NPGVAMATHK DDEERFFPSS GVLMFGKQGA
551 GKDNVDYSSV MLTSEEEIKT TNPVATEQYG VVADNLQQQN AAPIVGAVNS
601 QGALPGMVWQ NRDVYLQGPI WAKIPHTDGN FHPSPLMGGF GLKHPPPQIL
651 IKNTPVPADP PTTFSQAKLA SFITQYSTGQ VSVEIEWELQ KENSKRWNPE
701 IQYTSNYYKS TNVDFAVNTD GTYSEPRPIG TRYLTRNL
```

### Example of an Amino Acid Sequence of Wild-Type AAV11 Capsid Protein

```
(SEQ ID NO: 12)
    1 MAADGYLPDW LEDNLSEGIR EWWDLKPGAP KPKANQQKQD DGRGLVLPGY
  51 KYLGPFNGLD KGEPVNAADA AALEHDKAYD QQLKAGDNPY LRYNHADAEF
101 QERLQEDTSF GGNLGRAVFQ AKKRVLEPLG LVEEGAKTAP GKKRPLESPQ
151 EPDSSSGIGK KGKQPARKRL NFEEDTGAGD GPPEGSDISA MSSDIEMRAA
201 PGGNAVDAGQ GSDGVGNASG DWHCDSTWSE GKVTTTSTRT WVLPTYNNHL
251 YLRLGTTSSS NTYNGFSTPW GYFDFNRFHC HFSPRDWQRL INNNWGLRPK
301 AMRVKIFNIQ VKEVTTSNGE TTVANNLIST VQIFADSSYE LPYVMDAGQE
351 GSLPPFPNDV FMVPQYGYCG IVTGENQNQT DRNAFYCLEY FPSQMLRTGN
401 NFEMAYNFEK VPFHSMYAHS QSLDRLMNPL LDQYLWHLQS TTSGETLNQG
451 NAATTFGKIR SGDFAFYRKN WLPGPCVKQQ RESKTASQNY KIPASGGNAL
501 LKYDTHYTLN NRWSNIAPGP PMATAGPSDG DFSNAQLIFP GPSVTGNTTT
551 SANNLLFTSE EEIAATNPRD TDMFGQIADN NQNATTAPIT GNVTAMGVLP
601 GMVWQNRDIY YQGPIWAKIP HADGHFHPSP LIGGFGLKHP PPQIFIKNTP
651 VPANPATTFT AARVDSFITQ YSTGQVAVQI EWEIEKERSK RWNPEVQFTS
701 NYGNQSSMLW APDTTGKYTE PRVIGSRYLT NHL
```

### Example of an Amino Acid Sequence of Wild-Type AAV12 Capsid Protein

```
(SEQ ID NO: 13)
    1 MAADGYLPDW LEDNLSEGIR EWWALKPGAP QPKANQQHQD NGRGLVLPGY
  51 KYLGPFNGLD KGEPVNEADA AALEHDKAYD KQLEQGDNPY LKYNHADAEF
101 QQRLATDTSF GGNLGRAVFQ AKKRILEPLG LVEEGVKTAP GKKRPLEKTP
151 NRPTNPDSGK APAKKKQKDG EPADSARRTL DFEDSGAGDG PPEGSSSGEM
201 SHDAEMRAAP GGNAVEAGQG ADGVGNASGD WHCDSTWSEG RVTTTSTRTW
251 VLPTYNNHLY LRIGTTANSN TYNGFSTPWG YFDENRFHCH FSPRDWQRLI
301 NNNWGLRPKS MRVKIFNIQV KEVTTSNGET TVANNLISTV QIFADSTYEL
351 PYVMDAGQEG SFPPFPNDVF MVPQYGYCGV VTGKNQNQTD RNAFYCLEYF
401 PSQMLRTGNN FEVSYQFEKV PFHSMYAHSQ SLDRMMNPLL DQYLWHLQST
451 TTGNSLNQGT ATTTYGKITT GDFAYYRKNW LPGACIKQQK FSKNANQNYK
501 IPASGGDALL KYDTHTTLNG RWSNMAPGPP MATAGAGDSD FSNSQLIFAG
551 PNPSGNTTTS SNNLLFTSEE EIATTNPRDT DMFGQIADNN QNATTAPHIA
601 NLDAMGIVPG MVWQNRDIYY QGPIWAKVPH TDGHFHPSPL MGGFGLKHPP
651 PQIFIKNTPV PANPNITESA ARINSFLIQY STGQVAVQID WEIQKEHSKR
701 WNPEVQFTSN YGTQNSMLWA PDNAGNYHEL RAIGSRFLTH HL
```

### Example of an Amino Acid Sequence of Wild-Type AAVrh10 Capsid Protein

```
(SEQ ID NO: 14)
    1 MAADGYLPDW LEDNLSEGIR EWWDLKPGAP KPKANQQKQD DGRGLVLPGY
  51 KYLGPFNGLD KGEPVNAADA AALEHDKAYD QQLKAGDNPY LRYNHADAEF
101 QERLQEDTSF GGNLGRAVFQ AKKRVLEPLG LVEEGAKTAP GKKRPVEPSP
151 QRSPDSSTGI GKKGQQPAKK RLNFGQTGDS ESVPDPQPIG EPPAGPSGLG
201 SGTMAAGGGA PMADNNEGAD GVGSSSGNWH CDSTWLGDRV ITTSTRTWAL
251 PTYNNHLYKQ ISNGTSGGST NDNTYFGYST PWGYFDENRF HCHFSPRDWQ
301 RLINNNWGFR PKRLNFKLEN IQVKEVTQNE GTKTIANNLT STIQVFTDSE
351 YQLPYVLGSA HQGCLPPFPA DVFMIPQYGY LTLNNGSQAV GRSSFYCLEY
401 FPSQMLRTGN NFEFSYQFED VPFHSSYAHS QSLDRLMNPL IDQYLYYLSR
451 TQSTGGTAGT QQLLFSQAGP NNMSAQAKNW LPGPCYRQQR VSTTLSQNNN
501 SNFAWTGATK YHLNGRDSLV NPGVAMATHK DDEERFFPSS GVLMFGKQGA
551 GKDNVDYSSV MLTSEEEIKT TNPVATEQYG VVADNLQQQN AAPIVGAVNS
601 QGALPGMVWQ NRDVYLQGPI WAKIPHTDGN FHPSPLMGGF GLKHPPPQIL
651 IKNTPVPADP PTTFSQAKLA SFITQYSTGQ VSVEIEWELQ KENSKRWNPE
701 IQYTSNYYKS TNVDFAVNTD GTYSEPRPIG TRYLTRNL
```

### Example of a Nucleotide Sequence Encoding AAVrh74 Capsid Protein:

(SEQ ID NO: 15)
atggctgccgatggttatcttccagattggctcgaggacaacctct
ctgagggcattcgcgagtggtgggacctgaaacctggagccccga
aacccaaagccaaccagcaaaagcaggacaacggccggggtctgg
tgcttcctggctacaagtacctcggacccttcaacggactcgaca
agggggagcccgtcaacgcggcggacgcagcggccctcgagcacg
acaaggcctacgaccagcagctccaagcgggtgacaatccgtacc
tgcggtataatcacgccgacgccgagtttcaggagcgtctgcaag
aagatacgtcttttgggggcaacctcgggcgcgcagtcttccagg
ccaaaaagcgggttctcgaacctctgggcctggttgaatcgccgg
ttaagacggctcctggaaagaagagaccggtagagccatcacccc
agcgctctccagactcctctacgggcatcggcaagaaaggccagc
agcccgcaaaaaagagactcaattttgggcagactggcgactcag
agtcagtccccgaccctcaaccaatcggagaaccaccagcaggcc
cctctggtctgggatctggtacaatggctgcaggcggtggcgctc
caatggcagacaataacgaaggcgccgacggagtgggtagttcct
caggaaattggcattgcgattccacatggctgggcgacagagtca
tcaccaccagcacccgcacctgggccctgcccacctacaacaacc
acctctacaagcaaatctccaacgggacctcgggaggaagcacca
acgacaacacctacttcggctacagcaccccctgggggtattttg
acttcaacagattccactgccacttttcaccacgtgactggcagc
gactcatcaacaacaactggggattccggcccaagaggctcaact
tcaagctcttcaacatccaagtcaaggaggtcacgcagaatgaag
gcaccaagaccatcgccaataaccttaccagcacgattcaggtct
ttacggactcggaataccagctcccgtacgtgctcggctcggcgc
accagggctgcctgcctccgttcccggcggacgtcttcatgattc
ctcagtacgggtacctgactctgaacaatggcagtcaggctgtgg
gccggtcgtccttctactgcctggagtactttccttctcaaatgc
tgagaacgggcaacaactttgaattcagctacaacttcgaggacg
tgcccttccacagcagctacgcgcacagccagagcctggaccggc
tgatgaaccctctcatcgaccagtacttgtactacctgtcccgga
ctcaaagcacgggcggtactgcaggaactcagcagttgctatttt
ctcaggccgggcctaacaacatgtcggctcaggccaagaactggc
tacccggtccctgctaccggcagcaacgcgtctccacgacactgt
cgcagaacaacaacagcaactttgcctggacgggtgccaccaagt
atcatctgaatggcagagactctctggtgaatcctggcgttgcca
tggctacccacaaggacgacgaagagcgattttttccatccagcg
gagtcttaatgtttgggaaacagggagctggaaaagacaacgtgg
actatagcagcgtgatgctaaccagcgaggaagaaataaagacca
ccaacccagtggccacagaacagtacggcgtggtggccgataacc
tgcaacagcaaaacgccgctcctattgtaggggccgtcaatagtc
aaggagccttacctggcatggtgtggcagaaccgggacgtgtacc
tgcagggtcccatctgggccaagattcctcatacggacggcaact
ttcatccctcgccgctgatgggaggctttggactgaagcatccgc
ctcctcagatcctgattaaaaacacacctgttcccgccgatcctc
cgaccaccttcaatcaggccaagctggcttctttcatcacgcagt
acagtaccggtcaggtcagcgtggagatcgagtgggagctgcaga
aggagaacagcaaacgctggaacccagagattcagtacacttcca
actactacaaatctacaaatgtggactttgctgtcaatactgagg
gtacttattccgagcctcgccccattggcacccgttacctcaccc
gtaatctgtaa

In some embodiments, an AAV (e.g., AAVrh74) capsid protein comprises an amino acid deletion or substitution at a position corresponding to T265 of wild-type AAVrh74 (e.g., as shown in SEQ ID NO: 1). In some embodiments, an AAV (e.g., AAVrh74) capsid protein comprises an amino acid substitution at a position corresponding to Y447, T494, K547, N665, and/or Y733 of wild-type AAVrh74 capsid protein of SEQ ID NO: 1. In some embodiments, an AAV (e.g., AAVrh74) capsid protein comprises an amino acid deletion or substitution at a position corresponding to T265 of wild-type AAVrh74, and an amino acid substitution at a position corresponding to Y447, T494, K547, N665, and/or Y733 of wild-type AAVrh74 capsid protein of SEQ ID NO: 1.

In some embodiments, an AAV capsid protein comprises any one of the following amino acid substitutions: T265D, T265F, and T265G at a position corresponding to T265 of wild-type AAVrh74 (e.g., of sequence of SEQ ID NO: 1). In some embodiments, a capsid protein as provided herein has a deletion at a position corresponding to T265 of wild-type AAVrh74 (e.g., of sequence of SEQ ID NO: 1). In some embodiments, an amino acid at a position corresponding to T265 of wild-type AAVrh74 (e.g., of sequence of SEQ ID NO: 1) is A, R, N, D, C, Q, E, G, H, I, L, K, M, F, P, S, W, Y, or V. In some embodiments, an amino acid at a position corresponding to T265 of wild-type AAVrh74 (e.g., of sequence of SEQ ID NO: 1) is a negatively charged amino acid (e.g., D or E). In some embodiments, an amino acid at a position corresponding to T265 of wild-type AAVrh74 (e.g., of sequence of SEQ ID NO: 1) is an acidic polar amino acid (e.g., D or E). In some embodiments, a amino acid at a position corresponding to T265 of wild-type AAVrh74 (e.g., of sequence of SEQ ID NO: 1) is not neutral or polar.

In some embodiments, an AAV capsid protein comprises a deletion at a position corresponding to T265 of wild-type AAVrh74 (e.g., as shown in SEQ ID NO: 1). In some embodiments, an AAV capsid protein comprises a deletion at a position corresponding to T265 of wild-type AAVrh74 (e.g., as shown in SEQ ID NO: 1) and an amino acid substitution at a position corresponding to Y447 of wild-type AAVrh74 (e.g., as shown in SEQ ID NO: 1). In some embodiments, an AAV capsid protein comprises a deletion at a position corresponding to T265 of wild-type AAVrh74 (e.g., as shown in SEQ ID NO: 1) and an amino acid substitution at a position corresponding to T494 of wild-type AAVrh74 (e.g., as shown in SEQ ID NO: 1). In some embodiments, an AAV capsid protein comprises a deletion at a position corresponding to T265 of wild-type AAVrh74 (e.g., as shown in SEQ ID NO: 1) and an amino acid substitution at a position corresponding to K547 of wild-type AAVrh74 (e.g., as shown in SEQ ID NO: 1). In some embodiments, an AAV capsid protein comprises a deletion at a position corresponding to T265 of wild-type AAVrh74 (e.g., as shown in SEQ ID NO: 1) and an amino acid substitution at a position corresponding to N665 of wild-type AAVrh74 (e.g., as shown in SEQ ID NO: 1). In some embodiments, an AAV capsid protein comprises a deletion at a position corresponding to T265 of wild-type AAVrh74 (e.g., as shown in SEQ ID NO: 1) and an amino acid substitution at a position corresponding to Y733 of wild-type AAVrh74 (e.g., as shown in SEQ ID NO: 1). In some embodiments, an AAV capsid protein comprises a deletion at a position corresponding to T265 of wild-type AAVrh74 (e.g., as shown in SEQ ID NO: 1) and an amino acid substitution at a position corresponding to Y447 and Y733 of wild-type AAVrh74 (e.g., as shown in SEQ ID NO: 1). In some embodiments, an AAV capsid protein comprises a deletion at a position corresponding to T265 of wild-type AAVrh74 (e.g., as shown in SEQ ID NO: 1) and an amino acid substitution at a position corresponding to Y447, Y733, and T494 of wild-type AAVrh74 (e.g., as shown in SEQ ID NO: 1).

In some embodiments, an AAV capsid protein comprises an amino acid substitution at a position corresponding to T265 (e.g., T265D. T265G, or T265F) of wild-type AAVrh74 (e.g., as shown in SEQ ID NO: 1). In some embodiments, an AAV capsid protein comprises an amino acid substitution at a position corresponding to T265 (e.g., T265D. T265G, or T265F) of wild-type AAVrh74 (e.g., as shown in SEQ ID NO: 1) and an amino acid substitution at a position corresponding to Y447 of wild-type AAVrh74 (e.g., as shown in SEQ ID NO: 1). In some embodiments, an AAV capsid protein comprises an amino acid substitution at a position corresponding to T265 (e.g., T265D. T265G, or T265F) of wild-type AAVrh74 (e.g., as shown in SEQ ID NO: 1) and an amino acid substitution at a position corresponding to T494 of wild-type AAVrh74 (e.g., as shown in SEQ ID NO: 1). In some embodiments, an AAV capsid protein comprises an amino acid substitution at a position corresponding to T265 (e.g., T265D. T265G, or T265F) of wild-type AAVrh74 (e.g., as shown in SEQ ID NO: 1) and an amino acid substitution at a position corresponding to K547 of wild-type AAVrh74 (e.g., as shown in SEQ ID NO: 1). In some embodiments, an AAV capsid protein comprises an amino acid substitution at a position corresponding to T265 (e.g., T265D. T265G, or T265F) of wild-type AAVrh74 (e.g., as shown in SEQ ID NO: 1) and an amino acid substitution at a position corresponding to N665 of wild-type AAVrh74 (e.g., as shown in SEQ ID NO: 1). In some embodiments, an AAV capsid protein comprises an amino acid substitution at a position corresponding to T265 (e.g., T265D, T265G, or T265F) of wild-type AAVrh74 (e.g., as shown in SEQ ID NO: 1) and an amino acid substitution at a position corresponding to Y733 of wild-type AAVrh74 (e.g., as shown in SEQ ID NO: 1). In some embodiments, an AAV capsid protein comprises an amino acid substitution at a position corresponding to T265 (e.g., T265D. T265G, or T265F) of wild-type AAVrh74 (e.g., as shown in SEQ ID NO: 1) and an amino acid substitution at a position corresponding to Y447 and Y733 of wild-type AAVrh74 (e.g., as shown in SEQ ID NO: 1). In some embodiments, an AAV capsid protein comprises an amino acid substitution at a position corresponding to T265 (e.g., T265D. T265G, or T265F) of wild-type AAVrh74 (e.g., as shown in SEQ ID NO: 1) and an amino acid substitution at a position corresponding to Y447, Y733, and T494 of wild-type AAVrh74 (e.g., as shown in SEQ ID NO: 1).

In some embodiments, an amino acid substitution at a position corresponding to Y447 of wild-type AAVrh74 capsid protein (as shown e.g., in SEQ ID NO: 1) is Y447F. In some embodiments, an amino acid substitution at a position corresponding to Y733 of wild-type AAVrh74 capsid protein (as shown e.g., in SEQ ID NO: 1) is Y733F. In some embodiments, an amino acid substitution at a position corresponding to T494 of wild-type AAVrh74 capsid protein (as shown e.g., in SEQ ID NO: 1) is T494V. In some embodiments, an amino acid substitution at a position corresponding to K547 of wild-type AAVrh74 capsid protein (as shown e.g., in SEQ ID NO: 1) is K547R. In some embodiments, an amino acid substitution at a position corresponding to N665 of wild-type AAVrh74 capsid protein (as shown e.g., in SEQ ID NO: 1) is N665R. In some embodiments, an amino acid substitution at a position corresponding to Y733 of wild-type AAVrh74 capsid protein (as shown e.g., in SEQ ID NO: 1) is Y733F.

In some embodiments, an AAV capsid protein comprises an amino acid substitution at a position corresponding to T265 (e.g., T265D. T265G, or T265F) of wild-type AAVrh74 (e.g., as shown in SEQ ID NO: 1) and one or more (e.g., 1, 2, 3, 4, or 5) additional amino acid substitutions at one or more positions corresponding to Y447, Y733, T494, K547, and/or N665 of wild-type AAVrh74 (e.g., as shown in SEQ ID NO: 1). In some embodiments, the one or more additional amino acid substitutions correspond to Y447F, Y733F. T494V, K547R, and/or N665R of wild-type AAVrh74 capsid protein (as shown e.g., in SEQ ID NO: 1).

### Nucleic Acids Encoding AAV Capsid Proteins

Provided herein are nucleic acids encoding capsid proteins. A nucleic acid may comprise a sequence that encodes a capsid protein disclosed here (e.g., a capsid protein comprising one or more amino acid substitutions). A sequence encoding a capsid protein disclosed herein can be determined by one of ordinary skill in the art by known methods. A nucleic acid encoding a capsid protein may comprise a promoter or other regulatory sequence operably linked to the coding sequence. A nucleic acid encoding a capsid protein may be in the form of a plasmid, an mRNA, or another nucleic acid capable of being used by enzymes or machinery of a host cell to produce a capsid protein. Nucleic acids encoding capsid proteins as provided herein can be used to make AAV particles that can be used for delivering a gene to a cell. Methods of making AAV particles are known in the art. For example, see Scientific Reports volume 9, Article number: 13601 (2019); Methods Mol Biol. 2012; 798:267-284; and www.thermofisher.com/us/en/home/clinical/cell-gene-therapy/gene-therapy/aav-production-workflow.html.

### AAV Particles

Provided herein are AAV particles that comprise any of the AAV capsid proteins disclosed herein. AAV particles may be of any serotype (e.g., or serotype 1, serotype 2, serotype 3, serotype 4, serotype 5, serotype 6, serotype 7, serotype 8, serotype 9, serotype 10, serotype rh10, serotype 11, serotype 12, serotype 13, or serotype rh74). In some embodiments, an AAV particle as provided herein is empty. In some embodiments, an AAV particle as provided herein comprises a nucleic acid encapsidated by capsid protein. In some embodiments, a nucleic acid encapsidated by any one of the AAV particles provided herein comprises a gene of interest flaked by inverted terminal repeats (ITRs). In some embodiments, a nucleic acid encapsidated by any one of the AAV particles provided herein comprises a gene of interest and a regulatory element that is operably linked to the gene of interest.

In some embodiments, a gene of interest encodes a therapeutic molecule. A therapeutic molecule may be a an antibody, a peptibody, a growth factor, a clotting factor, a hormone, a membrane protein, a cytokine, a chemokine, an activating or inhibitory peptide acting on cell surface receptors or ion channels, a cell-permeant peptide targeting intracellular processes, a thrombolytic, an enzyme, a bone morphogenetic protein, a nuclease or other protein used for gene editing, an Fc-fusion protein, an anticoagulant, a nuclease, guide RNA or other nucleic acid or protein for gene editing, or any functional portion of any of these molecules.

In some embodiments, a therapeutic molecule, such as a therapeutic protein, is one that affects muscle function. For example, a therapeutic molecule may be a protein that is implicated in a muscular dystrophy. Non-limiting examples of proteins implicated in a muscular dystrophy are dystrophin, myotilin, lamin, caveolin, caplain-3, dysferlin, a sarcoglycan, AUF1, TCAP, TRIM32, FKRP, titin, acetylflucosamine epimerase, Desmin, LARGE, fukutin, an integrin, salenoprotein, a collagen, and plectin. Lovering et al. (Phys Ther. 2005 December; 85 (12): 1372-1388), provides examples of muscular dystrophies and implicated proteins that can be targeted for therapy.

A regulatory element refers to a nucleotide sequence or structural component of a nucleic acid which is involved in the regulation of expression of components of the nucleic acid vector (e.g., a gene of interest comprised therein). Regulatory elements include, but are not limited to, promoters, enhancers, silencers, insulators, response elements, initiation sites, termination signals, and ribosome binding sites.

Promoters include constitutive promoters, inducible promoters, tissue-specific promoters, cell type-specific promoters, and synthetic promoters. For example, a nucleic acid vector disclosed herein may include viral promoters or promoters from mammalian genes that are generally active in promoting transcription. Non-limiting examples of constitutive viral promoters include the Herpes Simplex virus (HSV), thymidine kinase (TK), Rous Sarcoma Virus (RSV), Simian Virus 40 (SV40), Mouse Mammary Tumor Virus (MMTV), Ad E1A and cytomegalovirus (CMV) promoters. Non-limiting examples of constitutive mammalian promoters include various housekeeping gene promoters, as exemplified by the β-actin promoter.

Inducible promoters or other inducible regulatory elements may also be used to achieve desired expression levels of a gene of interest (e.g., a protein or polypeptide of interest). Non-limiting examples of suitable inducible promoters include those from genes such as cytochrome P450 genes, heat shock protein genes, metallothionein genes, and hormone-inducible genes, such as the estrogen gene promoter. Another example of an inducible promoter is the tetVP16 promoter that is responsive to tetracycline.

Tissue-specific promoters or other tissue-specific regulatory elements are also contemplated herein. Non-limiting examples of such promoters that may be used include muscle-specific promoters.

Synthetic promoters are also contemplated herein. A synthetic promoter may comprise, for example, regions of known promoters, regulatory elements, transcription factor binding sites, enhancer elements, repressor elements, and the like.

In some embodiments, a gene of interest encodes a detectable molecule. A detectable molecule is one that can be detected in a sample of tissue or an organ or in a subject body by some imaging method. In some embodiments, a detectable molecule is a fluorescent, bioluminescent, radiolabeled, or enzymatic protein or functional peptide or functional polypeptide thereof.

Additional features of AAV particles, nucleic acid encapsidated in them, and capsid proteins are described in U.S. Patent Publication No. 2017/0356009, the contents of which are incorporated herein by reference in their entirety.

### Liver Detargeting

In some embodiments, the AAV particles comprising one or more amino acid deletions and/or substitutions in a capsid protein are liver detargeted. In some embodiments, an AAV particle that is liver detargeted is sequestered by the liver after being administered to a subject to a lesser degree than a corresponding wild-type AAV of the same serotype or a corresponding AAV not comprising the one or more amino acid deletions and/or substitutions. For example, administering of a composition comprising wild-type AAV (e.g., of serotype rh74) may result in sequestration by the liver of 50% of the particles in the composition, whereas administering of a composition comprising any one of the AAV particles comprising one or more amino acid deletions and/or substitutions in a capsid protein and of same serotype and same concentration and/or amount, may result in sequestration by the liver of less than 50% (e.g., 40, 30, 20, 10, or 5 or less %) of the particles in the composition.

In some embodiments, liver detargeting results in an increase in sequestration by a target organ or tissue (e.g., muscle tissue). In some embodiments, the target organ or tissue is not liver. For example, a composition comprising any one of the AAV particles disclosed herein may be sequestered by muscle tissue to a higher degree (e.g., 5, 10, 20, 30, 40, 50, 60, 70, 80, 90, 100, 200 or more % more) compared to a corresponding wild-type AAV of the same serotype or a corresponding AAV not comprising the one or more amino acid deletions and/or substitutions.

Liver detargeting or increase in sequestration by another tissue (e.g., muscle or brain) can be measured by sampling of the liver or tissue in question, e.g., by taking a biopsy. A biopsy can be assessed using various methods to detect and quantify the amount of AAV particles that were sequestered. For example, a tag may be measured, e.g., by a visualization method such as fluorescence. Another way to measure or quantify AAV is to measure the amount of capsid protein present or the amount of transgene delivered.

### Transduction Efficiency

In some embodiments, the AAV particles comprising one or more amino acid deletions and/or substitutions in a capsid protein have a higher transduction efficiency compared to a corresponding wild-type AAV particle of the same serotype or a corresponding AAV particle not comprising the one or more amino acid deletions and/or substitutions. Transduction efficiency of an AAV particle can be determined, for example, by comparing expression of a gene of interest in a cell following contacting the cell with the AAV particle. In some embodiments, transduction efficiency of an AAV particle as disclosed herein (e.g., an AAV particle comprising one or more amino acid substitutions or deletions) is higher than the transduction efficiency of a corresponding wild-type AAV particle or AAV particle that is of the same serotype but that does not have the amino acid deletions or substitutions. In some embodiments, the transduction efficiency of an AAV particle as disclosed herein is at least 5% higher (e.g., at least 10% higher, at least 15% higher, at least 20% higher, at least 25% higher, at least 30% higher, at least 35% higher, at least 40% higher, at least 50% higher, at least 60% higher, at least 70% higher, at least 80% higher, at least 90% higher, at least 100% higher, at least 150% higher, at least 200% higher, at least 250% higher, or more) than the transduction efficiency of a corresponding wild-type AAV particle or AAV particle that is of the same serotype but that does not have the amino acid deletions or substitutions. In some embodiments, the transduction efficiency of an AAV particle as disclosed herein is at least 1.5-fold higher (e.g., at least 2-fold higher, at least 2.5-fold higher, at least 3-fold higher, at least 3.5-fold higher, at least 4-fold higher, at least 4.5-fold higher, at least 5-fold higher, at least 5.5-fold higher, at least 6-fold higher, at least 6.5-fold higher, at least 7-fold higher, at least 7.5-fold higher, at least 8-fold higher, at least 8.5-fold higher, at least 9-fold higher, at least 9.5-fold higher, at least 10-fold higher, at least 10.5-fold higher, at least 11-fold higher, at least 11.5-fold higher, at least 12-fold higher, at least 12.5-fold higher, at least 13-fold higher, at least 13.5-fold higher, at least 14-fold higher, at least 14.5-fold higher, at least 15-fold higher, at least 15.5-fold higher, at least 16-fold higher, at least 16.5-fold higher, at least 17-fold higher, at least 17.5-fold higher, at least 18-fold higher, at least 18.5-fold higher, at least 19-fold higher, at least 19.5-fold higher, at least 20-fold higher, or more) than the transduction efficiency of a corresponding wild-type AAV particle or AAV particle that is of the same serotype but that does not have the amino acid deletions or substitutions. In some embodiments, transduction efficiency of an AAV particle as disclosed herein is not modified relative to a corresponding wild-type AAV particle or AAV particle that is of the same serotype but does that not have the amino acid deletions or substitutions.

### Pharmaceutical Compositions

Any one of the AAV particles, capsid proteins, or nucleic acids disclosed herein may be comprised within a pharmaceutical composition comprising a pharmaceutically-acceptable carrier or may be comprised within a pharmaceutically-acceptable carrier. The term “carrier” refers to a diluent, adjuvant, excipient, or vehicle with which the AAV particle, capsid protein, or nucleic acid is comprised or administered to a subject. Such pharmaceutical carriers can be sterile liquids, such as water and oils, including those of petroleum oil such as mineral oil, vegetable oil such as peanut oil, soybean oil, and sesame oil, animal oil, or oil of synthetic origin. Saline solutions and aqueous dextrose and glycerol solutions can also be employed as liquid carriers. Non-limiting examples of pharmaceutically acceptable carriers include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline, syrup, methylcellulose, ethylcellulose, hydroxypropylmethylcellulose, polyacrylic acids, lubricating agents (such as talc, magnesium stearate, and mineral oil), wetting agents, emulsifying agents, suspending agents, preserving agents (such as methyl-, ethyl-, and propyl-hydroxy-benzoates), and pH adjusting agents (such as inorganic and organic acids and bases), and solutions or compositions thereof. Other examples of carriers include phosphate buffered saline, HEPES-buffered saline, and water for injection, any of which may be optionally combined with one or more of calcium chloride dihydrate, disodium phosphate anhydrous, magnesium chloride hexahydrate, potassium chloride, potassium dihydrogen phosphate, sodium chloride, or sucrose. Other examples of carriers that might be used include saline (e.g., sterilized, pyrogen-free saline), saline buffers (e.g., citrate buffer, phosphate buffer, acetate buffer, and bicarbonate buffer), amino acids, urea, alcohols, ascorbic acid, phospholipids, proteins (for example, serum albumin), EDTA, sodium chloride, liposomes, mannitol, sorbitol, and glycerol. USP grade carriers and excipients are particularly useful for delivery of AAV particles to human subjects.

Typically, such compositions may contain at least about 0.1% of the therapeutic agent (e.g., AAV particle) or more, although the percentage of the active ingredient(s) may, of course, be varied and may conveniently be between about 1 or 2% and about 70% or 80% or more of the weight or volume of the total formulation. Naturally, the amount of therapeutic agent(s) (e.g., AAV particle) in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be designed.

### Methods of Use

According to some aspects, methods of contacting a cell with an AAV particle are provided herein. Methods of contacting a cell may comprise, for example, contacting a cell in a culture with a composition comprising an AAV particle. In some embodiments, contacting a cell comprises adding a composition comprising an AAV particle to the supernatant of a cell culture (e.g., a cell culture on a tissue culture plate or dish) or mixing a composition comprising an AAV particle with a cell culture (e.g., a suspension cell culture). In some embodiments, contacting a cell comprises mixing a composition comprising an AAV particle with another solution, such as a cell culture media, and incubating a cell with the mixture.

In some embodiments, contacting a cell with an AAV particle comprises administering a composition comprising an AAV particle to a subject or device in which the cell is located. In some embodiments, contacting a cell comprises injecting a composition comprising an AAV particle into a subject in which the cell is located. In some embodiments, contacting a cell comprises administering a composition comprising an AAV particle directly to a cell, or into or substantially adjacent to a tissue of a subject in which the cell is present.

Aspects of this disclosure provide a method comprising administering to a subject any one of the compositions comprising any one of the AAV particles disclosed herein.

In some embodiments, “administering” or “administration” means providing a material to a subject in a manner that is pharmacologically useful. In some embodiments, an AAV particle (e.g., comprised in a composition) is administered to a subject enterally. In some embodiments, an enteral administration of the essential metal element/s is oral. In some embodiments, an AAV particle is administered to the subject parenterally. In some embodiments, an AAV particle is administered to a subject subcutaneously, intraocularly, intravitreally, subretinally, intravenously (IV), intracerebro-ventricularly, intramuscularly, intrathecally (IT), intracisternally, intraperitoneally, via inhalation, topically, or by direct injection to one or more cells, tissues, or organs. In some embodiments, an AAV particle is administered to the subject by injection into the hepatic artery or portal vein.

In some embodiments, a composition of AAV particles is administered to a subject to treat a disease or condition. To “treat” a disease as the term is used herein, means to reduce the frequency or severity of at least one sign or symptom of a disease or disorder experienced by a subject. The compositions described above or elsewhere herein are typically administered to a subject in an effective amount, that is, an amount capable of producing a desirable result. The desirable result will depend upon the active agent being administered. For example, an effective amount of rAAV particles may be an amount of the particles that are capable of transferring an expression construct to a host organ, tissue, or cell. A therapeutically acceptable amount may be an amount that is capable of treating a disease, e.g., a muscular dystrophy. As is well known in the medical and veterinary arts, dosage for any one subject depends on many factors, including the subject's size, body surface area, age, the particular composition to be administered, the active ingredient(s) in the composition, time and route of administration, general health, and other drugs being administered concurrently.

In some embodiments, a composition comprising any one of the particles disclosed herein comprises at least 2 times (e.g., 2-200 times, 2-4 times, 2-10 times, 5-10 times, 2-20 times, 10-20 times, 10-50 times, 20-50 times, 50-100 times, 50-200 times or more) less AAV particles compared to a composition of wild-type AAV particles or AAV particles not comprising amino acid substitutions would have to be to achieve the same delivery to a target tissue. For example, if 10¹⁴ particles of a wild-type AAVrh74 or an AAVrh74 particle with Y447+Y733F mutation but not T265D would have to be administered to achieve delivery of 40% of it to muscle tissue, then less than 10¹⁴ particles (e.g., 10¹³ particles or 10¹² particles) having Y447+Y733F and T265D would have to be administered to achieve the same delivery to muscle tissue. In some embodiments, a composition comprising any one of the particles disclosed herein for administration to a subject to target a non-liver tissue (e.g., muscle) comprises at least 10% less AAV particles of the same serotype without the mutations or deletions.

In some embodiments, a cell disclosed herein is a cell isolated or derived from a subject. In some embodiments, a cell is a mammalian cell (e.g., a cell isolated or derived from a mammal). In some embodiments, a cell is a human cell. In some embodiments, a cell is isolated or derived from a particular tissue of a subject, such as muscle tissue. In some embodiments, a cell is a muscle cell. In some embodiments, a cell is a skeletal muscle cell or a smooth muscle cell. In some embodiments, a cell is in vitro. In some embodiments, a cell is ex vivo. In some embodiments, a cell is in vivo. In some embodiments, a cell is within a subject (e.g., within a tissue or organ of a subject). In some embodiments, a cell is a primary cell. In some embodiments, a cell is from a cell line (e.g., an immortalized cell line). In some embodiments a cell is a cancer cell or an immortalized cell.

In some embodiments, the concentration of AAV particles administered to a subject may be on the order ranging from 10⁶ to 10¹⁵ particles/ml or 10³ to 10¹⁶ particles/ml, or any values therebetween for either range, such as for example, about 10⁶, 10⁷, 10⁸, 10⁹, 10¹⁰, 10¹¹, 10¹², 10¹³, 10¹⁴ or 10¹⁵ particles/ml. In some embodiments, AAV particles of a higher concentration than 10¹³ particles/ml are administered. In some embodiments, the concentration of AAV particles administered to a subject may be on the order ranging from 10⁶ to 10¹⁴ vector genomes (vgs)/ml or 10³ to 10¹⁵ vgs/ml, or any values therebetween for either range (e.g., 10⁶, 10⁷, 10⁸, 10⁹, 10¹⁰, 10¹¹, 10¹², 10¹³, or 10¹⁴ vgs/ml). In some embodiments, AAV particles of higher concentration than 10¹³ vgs/ml are administered. The AAV particles can be administered as a single dose, or divided into two or more administrations as may be required to achieve therapy of the particular disease or disorder being treated. In some embodiments, 0.0001 ml to 10 ml are delivered to a subject. In some embodiments, the number of AAV particles administered to a subject may be on the order ranging from 10⁶-10¹⁴ vgs/kg body mass of the subject, or any values therebetween (e.g., 10⁶, 10⁷, 10⁸, 10⁹, 10¹⁰, 10¹¹, 10¹², 10¹³, or 10¹⁴ vgs/kg). In some embodiments, the dose of AAV particles administered to a subject may be on the order ranging from 10¹²-10¹⁴ vgs/kg. In some embodiments, the volume of AAVrh74 composition delivered to a subject (e.g., via one or more routes of administration as described herein) is 0.0001 ml to 10 ml.

In some embodiments, a composition disclosed herein (e.g., comprising an AAV particle) is administered to a subject once. In some embodiments, the composition is administered to a subject multiple times (e.g., twice, three times, four times, five times, six times, or more). Repeated administration to a subject may be conducted at a regular interval (e.g., daily, every other day, twice per week, weekly, twice per month, monthly, every six months, once per year, or less or more frequently) as necessary to treat (e.g., improve or alleviate) one or more symptoms of a disease, disorder, or condition in the subject.

### Subjects

Aspects of the disclosure relate to methods for use with a subject, such as human or non-human primate subjects; with a host cell in situ in a subject; or with a host cell derived from a subject (e.g., ex vivo or in vitro). Non-limiting examples of non-human primate subjects include macaques (e.g., cynomolgus or rhesus macaques), marmosets, tamarins, spider monkeys, owl monkeys, vervet monkeys, squirrel monkeys, baboons, gorillas, chimpanzees, and orangutans. In some embodiments, the subject is a human subject. Other exemplary subjects include domesticated animals such as dogs and cats; livestock such as horses, cattle, pigs, sheep, goats, and chickens; and other animals such as mice, rats, guinea pigs, and hamsters.

In some embodiments, the subject has or is suspected of having a disease or disorder that may be treated with gene therapy. In some embodiments, the subject has or is suspected of having a muscle disease or disorder. A muscle disease or disorder is typically characterized by one or more mutation(s) in the genome that results in abnormal structure or function of one or more proteins associated with muscle development, health, maintenance and/or function. Exemplary muscle disease and disorders include amyotrophic lateral sclerosis, Charcot-Marie-Tooth disease, multiple sclerosis, muscular dystrophy (e.g., Duchenne muscular dystrophy, facioscapulohumeral muscular dystrophy, Becker muscular dystrophy, or limb-girdle muscular dystrophy (LGMD) such as LGMD type 1 or LGMD type 2), myasthenia gravis, myopathy (e.g., X-linked myotubular myopathy), myositis, peripheral neuropathy, or spinal muscular atrophy. Muscle diseases and disorders can be characterized and identified, e.g., through laboratory tests and/or evaluation by a clinician. In some embodiments, the subject has or is suspected of having a disease involving muscle cells (e.g., a disease caused by a defect, such as a genetic mutation, in one or more muscle cells or genes associated therewith). In some embodiments, a nucleic acid isolated or derived from the subject (e.g., genomic DNA, mRNA, or cDNA from the subject) is identified via sequencing (e.g., Sanger or next-generation sequencing) to comprise a mutation (e.g., in a gene associated with muscle development, health, maintenance, or function).

In some embodiments, a gene associated with muscle development, health, maintenance, or function is dystrophin/DMD, SCN4A, DMPK, ACTA, TPM3, TPM2, TNNT1, CFL2, KBTBD13, KLHL30, KKLHL3, KLHL41, LMOD3, MYPN, MTM1, nebulin, DNM2, TTN, RYR1, MYH7, TK2, GAA (α-glucosidase), CIC1, LMNA, CAV3, DNAJB6, TRIM32, desmin, LAMA2, COL6A1, COL6A2, COL6A3, or DUX4. In some embodiments the gene is dystrophin (DMD) or MTM1. In some embodiments, the gene is a gene in which mutations have been shown to cause limb-girdle muscular dystrophy (e.g., LGMD1 or LGMD2), such as MYOT, LMNA, CAV3, DNAJB6, DES, TNP03, HNRNPDL, CAPN3, DYSF, SGCG, SGCA, SGCB, SGCD, AUF1, TCAP, TRIM32, FKRP, TTN, POMT1, ANO5, FKTN, POMT2, POMGnT1, DAG1, PLEC1, DES, TRAPPC11, GMPPB, ISPD, GAA, LIMS2, BVES, or TOR1A1P1. In some embodiments, a subject comprises a mutant form of one or more genes associated with muscle development, health, maintenance or function. In some embodiments, methods disclosed herein provide a cell (e.g., a muscle cell) of a subject with a functional form of a gene associated with muscle development, health, maintenance, or function.

### EXAMPLES

### Example 1: Strategy to Detarget AAVrh74 Particles from the Liver and Improve Availability and Transduction Efficiency in Muscle Cells

A difficulty in using AAV particles to deliver a gene to a target tissue other than the liver is that a substantial portion of particles administered to a subject can be sequestered by the subject's liver, thus reducing the number of particles available to target other organs or tissues, such as muscle.

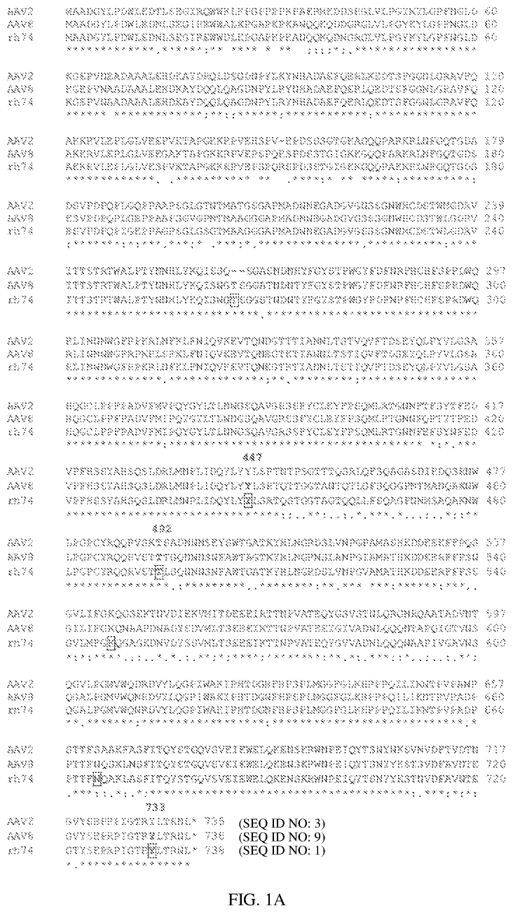

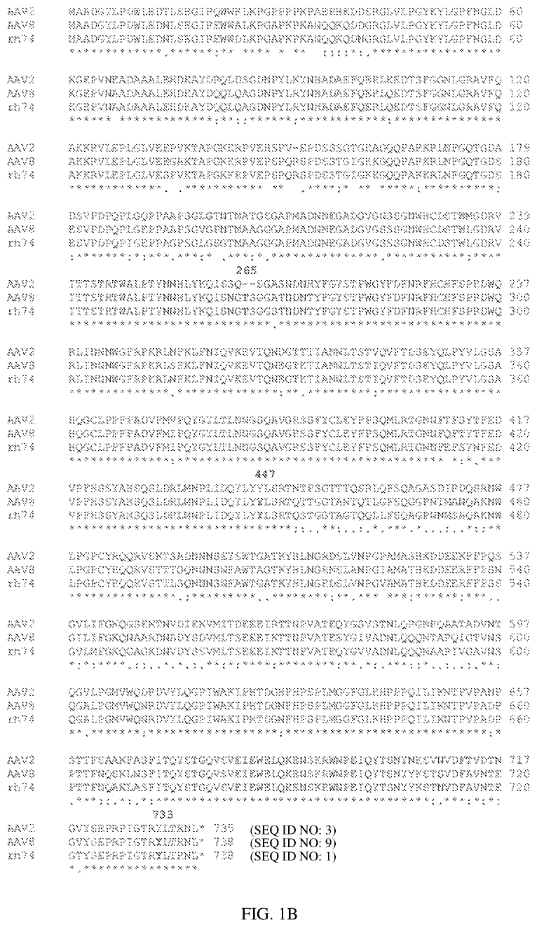

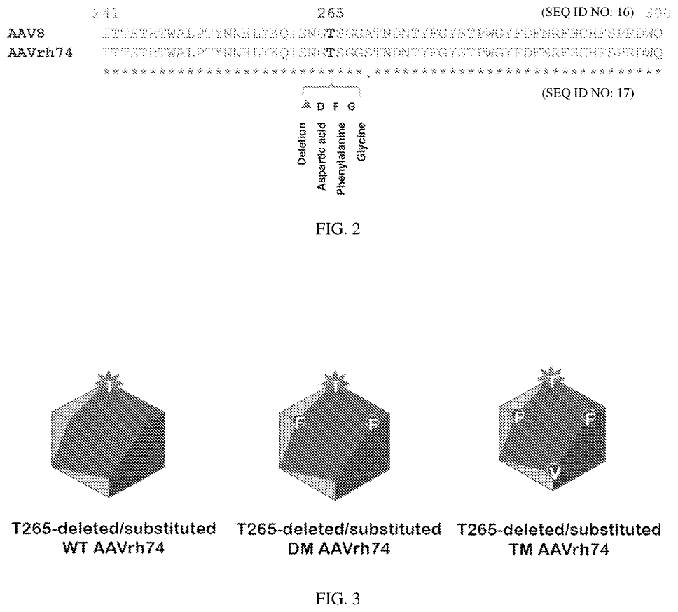

AAV8 is known to have a high tropism for the liver. Further, a single amino acid insertion in AAV-KP1 lead to greater transduction in hepatocytes (see Mol Ther Meth Clin Dev, 21:607-620, 2021). AAVrh74, while having a tropism for muscle cells, also get sequestered by the liver. By comparing the capsid sequences of AAVrh74 to that of AAV8, it was determined that deletion or substitution of T265 in rh74 will lead to liver detargeting. See FIGS. 1B and 2. Moreover, substitution of one or more of Y447F, Y733F, and T494V along with a deletion or substitution of T265 (see FIG. 1A for alignment) as shown in FIG. 3 will lead to a greater availability of administered AAVrh74 to a subject to target muscle cells/tissue and improve transduction efficiency in muscle cells/tissue.

### Example 2: Modified AAVrh74 Particles Achieve Liver Detargeting and Improve Muscle Delivery

To test the effect of AAVrh74 particle modifications on detargeting particles from the liver and increasing their delivery to muscle, ssAAVrh74 particles were prepared, comprising a nucleic acid vector encoding firefly luciferase (Fluc).

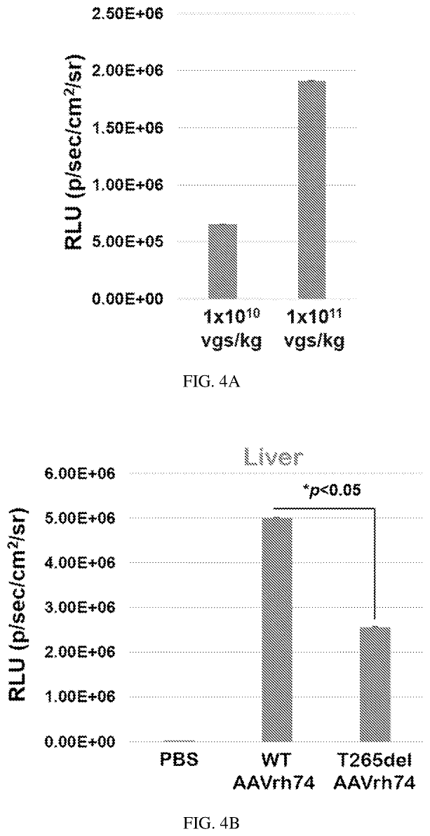

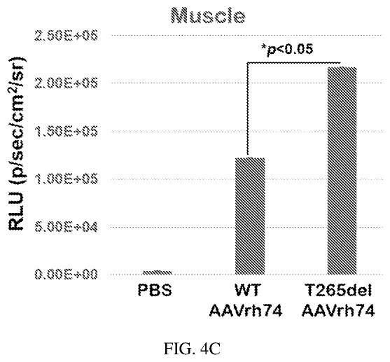

First, wild-type (WT) ssAAVrh74-Fluc particles were administered intravenously to C57BL6/J mice in quantities of 1×10¹⁰ or 1×10¹¹ viral genomes (vgs) per mouse. Two weeks after the particles were administered, whole body bioluminescence imaging was conducted. The results demonstrated abundant transduction of mouse liver by the WT ssAAVrh74-Fluc particles. The bioluminescence signal quantified from the imaging is shown in FIG. 4A. Mice administered 1×10¹⁰ vgs/mouse showed an average of approximately 6.5×10⁵ RLU (p/sec/cm²/sr), and mice administered 1×10¹¹ vgs/mouse showed an average of approximately 1.9×10⁶ RLU (p/sec/cm²/sr).

Next. WT ssAAVrh74-Fluc particles and ssAAVrh74-Fluc particles with a deletion of T265 (“T265del”) were prepared and administered intravenously to C57BL6/J mice in quantities of 1×10¹² vgs/mouse (n=5). Two weeks after the particles were administered, whole body bioluminescence imaging was performed. Bioluminescence was quantified in regions of interest comprising the liver (FIG. 4B) and the muscle (FIG. 4C) of the mice. The results demonstrate that T265del-ssAAVrh74-Fluc particles were significantly detargeted from the liver (P<0.05) relative to WT ssAA Vrh74-Fluc particles. Mice administered WT-ssAA Vrh74-Fluc particles showed an average of approximately 5×10⁶ RLU (p/sec/cm²/sr) in the liver, whereas mice administered T265del-ssAAVrh74-Fluc particles showed an average of approximately 2.5×10⁶ RLU (p/sec/cm²/sr) in the liver. Furthermore, the results demonstrate that the transduction efficiency of the T265del-ssAAVrh74-Fluc particles was significantly increased in muscle (P<0.05) relative to the WT ssAAVrh74-Fluc particles. Mice administered WT-ssAAVrh74-Fluc particles showed an average of approximately 1.2×10⁵ RLU (p/sec/cm²/sr) in the muscle, whereas mice administered T265del-ssAA Vrh74-Fluc particles showed an average of approximately 2.2×10⁵ RLU (p/sec/cm²/sr) in the muscle.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or.” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of.” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B”, the disclosure also contemplates the alternative embodiments “a composition consisting of A and B” and “a composition consisting essentially of A and B”.

What is claimed is:

1. An AAVrh74 capsid protein comprising an amino acid substitution or deletion at a position corresponding to T265 of wild-type AAVrh74 capsid protein of SEQ ID NO: 1.

2. The AAVrh74 capsid protein of claim 1, further comprising an amino acid substitution at a position corresponding to Y447, T494, K547, N665, and/or Y733 of wild-type AAVrh74 capsid protein of SEQ ID NO: 1.

3. The AAVrh74 capsid protein of claim 1 or claim 2, wherein the amino acid corresponding to T265 of wild-type AAVrh74 capsid protein of SEQ ID NO: 1 is deleted.

4. The AAVrh74 capsid protein of claim 1 or claim 2, wherein the amino acid corresponding to T265 of wild-type AAVrh74 capsid protein of SEQ ID NO: 1 is substituted.

5. The AAVrh74 capsid protein of claim 4, wherein substitution at the amino acid position corresponding to T265 of wild-type AAVrh74 capsid protein of SEQ ID NO: 1 is T265D, T265F, or T265G.

6. The AAVrh74 capsid protein of claim 1, wherein the capsid protein comprises an amino acid substitution or deletion at a position corresponding to T265 and amino acid substitutions at positions corresponding to:
(a) Y447 and Y733; or
(b) Y447, Y733, and T494
of wild-type AAVrh74 capsid protein of SEQ ID NO: 1.

7. The AAVrh74 capsid protein of claim 6, wherein the capsid protein comprises a deletion or one of the following substitutions at a position corresponding to T265: T265D, T265F, and T265G, and substitutions corresponding to:
(a) Y447F and Y733F; or
(b) Y447F, Y733F, and T494T
of wild-type AAVrh74 capsid protein of SEQ ID NO: 1.

8. A nucleic acid encoding the AAVrh74 capsid protein of any one of the preceding claims.

9. An AAV particle comprising the AAVrh74 capsid protein of any one of claims 1-7, and a nucleic acid comprising a gene of interest.

10. The AAV particle of claim 9, wherein the nucleic acid further comprising a muscle-specific promoter.

11. The AAV particle of claim 9 or claim 10, wherein the gene of interest encodes a therapeutic protein.

12. The AAV particle of claim 11, wherein the therapeutic protein is dystrophin, myotilin, lamin, caveolin, caplain-3, dysferlin, a sarcoglycan, AUF1, TCAP, TRIM32, FKRP, titin, acetylflucosamine epimerase, Desmin, LARGE, fukutin, an integrin, salenoprotein, a collagen, plectin, or a functional fragment thereof.

10. A composition comprising the AAV particle of any one of claims 9-12 and a pharmaceutically acceptable carrier.

11. A method comprising administering to a subject the composition of claim 10.

12. The method of claim 11, wherein the subject is a human subject.

13. The method of any claim 11 or 12, wherein the subject suffers from or is at risk of suffering from a muscular dystrophy.


SEQUENCE LISTING (17 sequences)

SEQ ID NO: 1 - protein, 738 aa, unidentified
  source 1..738: note = Adeno-associated virus serotype rh74
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 2 - protein, 736 aa, unidentified
  source 1..736: note = Adeno-associated virus serotype 1
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 3 - protein, 735 aa, unidentified [sequence omitted]

SEQ ID NO: 4 - protein, 736 aa, unidentified
  source 1..736: note = Adeno-associated virus serotype 3
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKGAVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVRGVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTGTVNHQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 5 - protein, 734 aa, unidentified
  source 1..734: note = Adeno-associated virus serotype 4
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 6 - protein, 724 aa, unidentified
  source 1..724: note = Adeno-associated virus serotype 5
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 7 - protein, 736 aa, unidentified
  source 1..736: note = Adeno-associated virus serotype 6
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 8 - protein, 737 aa, unidentified
  source 1..737: note = Adeno-associated virus serotype 7
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 9 - protein, 738 aa, unidentified
  source 1..738: note = Adeno-associated virus serotype 8
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 10 - protein, 736 aa, unidentified
  source 1..736: note = Adeno-associated virus serotype 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 11 - protein, 738 aa, unidentified
  source 1..738: note = Adeno-associated virus serotype 10
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 12 - protein, 733 aa, unidentified
  source 1..733: note = Adeno-associated virus serotype 11
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPLESPQEPDSSSGIGKKGKQPARKRLNFEEDTGAGDGPPEGSDTSAMSSDIEMRAAPGGNAVDAGQGSDGVGNASGDWHCDSTWSEGKVTTTSTRTWVLPTYNNHLYLRLGTTSSSNTYNGFSTPWGYFDFNRFHCHFSPRDWQRLINNNWGLRPKAMRVKIFNIQVKEVTTSNGETTVANNLTSTVQIFADSSYELPYVMDAGQEGSLPPFPNDVFMVPQYGYCGIVTGENQNQTDRNAFYCLEYFPSQMLRTGNNFEMAYNFEKVPFHSMYAHSQSLDRLMNPLLDQYLWHLQSTTSGETLNQGNAATTFGKIRSGDFAFYRKNWLPGPCVKQQRFSKTASQNYKIPASGGNALLKYDTHYTLNNRWSNIAPGPPMATAGPSDGDFSNAQLIFPGPSVTGNTTTSANNLLFTSEEEIAATNPRDTDMFGQIADNNQNATTAPITGNVTAMGVLPGMVWQNRDIYYQGPIWAKIPHADGHFHPSPLIGGFGLKHPPPQIFIKNTPVPANPATTFTAARVDSFITQYSTGQVAVQIEWEIEKERSKRWNPEVQFTSNYGNQSSMLWAPDTTGKYTEPRVIGSRYLTNHL

SEQ ID NO: 13 - protein, 742 aa, unidentified
  source 1..742: note = Adeno-associated virus serotype 12
MAADGYLPDWLEDNLSEGIREWWALKPGAPQPKANQQHQDNGRGLVLPGYKYLGPFNGLDKGEPVNEADAAALEHDKAYDKQLEQGDNPYLKYNHADAEFQQRLATDTSFGGNLGRAVFQAKKRILEPLGLVEEGVKTAPGKKRPLEKTPNRPTNPDSGKAPAKKKQKDGEPADSARRTLDFEDSGAGDGPPEGSSSGEMSHDAEMRAAPGGNAVEAGQGADGVGNASGDWHCDSTWSEGRVTTTSTRTWVLPTYNNHLYLRIGTTANSNTYNGFSTPWGYFDFNRFHCHFSPRDWQRLINNNWGLRPKSMRVKIFNIQVKEVTTSNGETTVANNLTSTVQIFADSTYELPYVMDAGQEGSFPPFPNDVFMVPQYGYCGVVTGKNQNQTDRNAFYCLEYFPSQMLRTGNNFEVSYQFEKVPFHSMYAHSQSLDRMMNPLLDQYLWHLQSTTTGNSLNQGTATTTYGKITTGDFAYYRKNWLPGACIKQQKFSKNANQNYKIPASGGDALLKYDTHTTLNGRWSNMAPGPPMATAGAGDSDFSNSQLIFAGPNPSGNTTTSSNNLLFTSEEEIATTNPRDTDMFGQIADNNQNATTAPHIANLDAMGIVPGMVWQNRDIYYQGPIWAKVPHTDGHFHPSPLMGGFGLKHPPPQIFIKNTPVPANPNTTFSAARINSFLTQYSTGQVAVQIDWEIQKEHSKRWNPEVQFTSNYGTQNSMLWAPDNAGNYHELRAIGSRFLTHHL

SEQ ID NO: 14 - protein, 738 aa, unidentified; identical to SEQ ID NO: 11
  source 1..738: note = Adeno-associated virus serotype rh10

SEQ ID NO: 15 - DNA, 2217 bp, unidentified
  source 1..2217: mol_type = genomic DNA; note = Adeno-associated virus serotype rh74
atggctgccgatggttatcttccagattggctcgaggacaacctctctgagggcattcgcgagtggtgggacctgaaacctggagccccgaaacccaaagccaaccagcaaaagcaggacaacggccggggtctggtgcttcctggctacaagtacctcggacccttcaacggactcgacaagggggagcccgtcaacgcggcggacgcagcggccctcgagcacgacaaggcctacgaccagcagctccaagcgggtgacaatccgtacctgcggtataatcacgccgacgccgagtttcaggagcgtctgcaagaagatacgtcttttgggggcaacctcgggcgcgcagtcttccaggccaaaaagcgggttctcgaacctctgggcctggttgaatcgccggttaagacggctcctggaaagaagagaccggtagagccatcaccccagcgctctccagactcctctacgggcatcggcaagaaaggccagcagcccgcaaaaaagagactcaattttgggcagactggcgactcagagtcagtccccgaccctcaaccaatcggagaaccaccagcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgcacctgggccctgcccacctacaacaaccacctctacaagcaaatctccaacgggacctcgggaggaagcaccaacgacaacacctacttcggctacagcaccccctgggggtattttgacttcaacagattccactgccacttttcaccacgtgactggcagcgactcatcaacaacaactggggattccggcccaagaggctcaacttcaagctcttcaacatccaagtcaaggaggtcacgcagaatgaaggcaccaagaccatcgccaataaccttaccagcacgattcaggtctttacggactcggaataccagctcccgtacgtgctcggctcggcgcaccagggctgcctgcctccgttcccggcggacgtcttcatgattcctcagtacgggtacctgactctgaacaatggcagtcaggctgtgggccggtcgtccttctactgcctggagtactttccttctcaaatgctgagaacgggcaacaactttgaattcagctacaacttcgaggacgtgcccttccacagcagctacgcgcacagccagagcctggaccggctgatgaaccctctcatcgaccagtacttgtactacctgtcccggactcaaagcacgggcggtactgcaggaactcagcagttgctattttctcaggccgggcctaacaacatgtcggctcaggccaagaactggctacccggtccctgctaccggcagcaacgcgtctccacgacactgtcgcagaacaacaacagcaactttgcctggacgggtgccaccaagtatcatctgaatggcagagactctctggtgaatcctggcgttgccatggctacccacaaggacgacgaagagcgattttttccatccagcggagtcttaatgtttgggaaacagggagctggaaaagacaacgtggactatagcagcgtgatgctaaccagcgaggaagaaataaagaccaccaacccagtggccacagaacagtacggcgtggtggccgataacctgcaacagcaaaacgccgctcctattgtaggggccgtcaatagtcaaggagccttacctggcatggtgtggcagaaccgggacgtgtacctgcagggtcccatctgggccaagattcctcatacggacggcaactttcatccctcgccgctgatgggaggctttggactgaagcatccgcctcctcagatcctgattaaaaacacacctgttcccgccgatcctccgaccaccttcaatcaggccaagctggcttctttcatcacgcagtacagtaccggtcaggtcagcgtggagatcgagtgggagctgcagaaggagaacagcaaacgctggaacccagagattcagtacacttccaactactacaaatctacaaatgtggactttgctgtcaatactgagggtacttattccgagcctcgccccattggcacccgttacctcacccgtaatctgtaa

SEQ ID NO: 16 - protein, 60 aa, unidentified
  source 1..60: note = Adeno-associated virus serotype 8
ITTSTRTWALPTYNNHLYKQISNGTSGGATNDNTYFGYSTPWGYFDFNRFHCHFSPRDWQ

SEQ ID NO: 17 - protein, 60 aa, unidentified
  source 1..60: note = Adeno-associated virus serotype rh74
ITTSTRTWALPTYNNHLYKQISNGTSGGSTNDNTYFGYSTPWGYFDFNRFHCHFSPRDWQ